(19) United States
(12) Patent Application Publication
(10) Pub. No.: US 2022/0218950 A1
(43) Pub. Date: Jul. 14, 2022

(54) PRESSURE RELIEVING DRESSING FOR PERIPHERAL INTRAVENOUS LINE

(71) Applicant: Taylor Alexandra Vozza, Sparta, NJ (US)

(72) Inventor: Taylor Alexandra Vozza, Sparta, NJ (US)

(21) Appl. No.: 17/453,921

(22) Filed: Nov. 8, 2021

Related U.S. Application Data
(60) Provisional application No. 63/199,553, filed on Jan. 8, 2021.

(51) Int. Cl. A61M 25/02; A61M 5/14; A61F 13/02

(52) U.S. Cl. CPC A61M 25/02; A61M 5/14; A61F 13/0266; A61F 2013/00412; A61F 13/025; A61F 13/0243; A61M 2025/0266; A61F 13/0233

(57) ABSTRACT

A skin protection and catheter securement dressing includes a body, a foam pad, a liner tab, a frame, and an adhesive strip. The body includes a plastic film with a transparent window, an adhesive surface, a recessed portion along one edge, and a second surface, covered on the perimeter by the frame to which the adhesive strip is attached. The adhesive surface is covered by release liner strips. The pad has an adhesive surface and an end hinged to the dressing body adhesive surface. A method includes preparing an IV site; placing a catheter; connecting an IV line; removing the liner tab; placing the foam pad under the hub; removing the liner strips; centering the IV line; adhering the film over the hub; removing the adhesive strip from the frame; removing the frame from the film; and adhering the adhesive strip over the IV line.

### CROSS-REFERENCE TO RELATED APPLICATION

This application claims the benefit of priority of U.S. provisional application No. 63/199,553, filed Jan. 8, 2021, the contents of which are herein incorporated by reference.

### BACKGROUND OF THE INVENTION

The present invention relates to dressings for peripheral intravenous lines and, more particularly, to pressure relieving dressings.

An intravenous catheter may be placed in a small vessel or vein in the body for venous access to administer medications and/or fluids. Invasive catheters (whether peripheral or central) cause pressure on the skin, resulting in breakdown and damage of the skin. The more fluid the patient receives, the more edematous the patient's skin becomes. The lines pressing into the skin cause bruises, indentations, and constant pain. In the medical field, substantial time is spent during lifesaving procedures to place protective devices under the catheter line while trying to also place a securing dressing on top. The chances of losing the line are very high through this multi-step process. The protective devices put the dressing at risk of moving and becoming lost or falling off.

There is no all-in-one device on the market. Currently available devices secure the line, but do not have protective barriers. They are heavy, bulky, and require multiple hands to place and secure the dressing.

As can be seen, there is a need for a device that both protects the patient's skin from pressure and secures a catheter line in place.

### SUMMARY OF THE INVENTION

The present invention provides an all-in-one skin protection and intravenous (IV) securement device that may be placed by one individual. The device has a soft protection component for placement underneath the catheter and a transparent securement component for placement over the catheter line to secure it in place. The soft material creates a barrier between the skin and the catheter line. Having both components attached together allows better catheter securement. This securement device relieves the pressure of the catheter line, alleviating pain and preventing bruises and skin breakdown.

In one aspect of the present invention, a skin protection and intravenous catheter securement dressing assembly is provided, comprising: a dressing body comprising a plastic film with a transparent window, a first adhesive surface, and a second surface, said plastic film having a recessed portion midway along one edge; two juxtaposed removable release liner strips covering the first adhesive surface; a foam pad with a second adhesive surface, a fixed end thereof integrally connected to the first adhesive surface of the dressing body, e.g., by a living hinge; a removable release liner tab covering the second adhesive surface; a removable release liner frame covering a perimeter of the second surface; and an adhesive strip removably attached to the removable release liner frame.

In another aspect of the present invention, a method for securing an intravenous catheter to a subject with a pressure relieving dressing is provided, comprising: preparing an intravenous (IV) site on the subject; placing an IV catheter with hub at the IV site; connecting an IV line to the hub; providing the skin protection and intravenous catheter securement dressing assembly; removing the removable release liner tab; placing the foam pad on the subject under the hub; removing the two juxtaposed removable release liner strips sequentially; centering the IV line in the recessed portion; adhering the plastic film on the IV site over the hub; removing the adhesive strip from the removable release liner frame; removing the removable release liner frame from the plastic film; and adhering the adhesive strip to the subject over the IV line.

These and other features, aspects and advantages of the present invention will become better understood with reference to the following drawings, description, and claims.

### BRIEF DESCRIPTION OF THE DRAWINGS

FIG. 1 is a perspective view of an IV dressing according to an embodiment of the present invention, shown in use;

FIG. 2 is a top perspective view thereof;

FIG. 3 is a bottom perspective view thereof;

FIG. 4 is an exploded view thereof;

FIG. 5 is a sectional view thereof, taken along line 5-5 of FIG. 1; and

FIG. 6 is a flow chart of an installation method according to an embodiment of the present invention.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is a pressure relieving dressing and intravenous catheter securement dressing assembly comprising a protective barrier component and a top transparent component. The protective barrier creates space between the catheter and the patient's skin adjacent to the insertion point. The transparent component holds the catheter in place and provides constant visibility of the subject's insertion site for continuous line and skin assessment. Together, both components allow medical personnel to protect the skin and secure the device in place quickly, safely, and easily.

The material used for the protective barrier under the catheter is not particularly limited and may be selected based on price, usage, etc. For example, the protective barrier may be made from a lightweight polymeric foam.

After the catheter line, a medical article, is placed, the provider may hold the line in one hand, pull back a first part of the dressing covering, and place the protective barrier underneath the catheter hub, against the patient's skin. The provider may place the transparent dressing over the catheter, remove a second part of the dressing covering, and secure the line.

In some embodiments, the transparent top catheter dressing is attached to the internal side of the protective barrier on one edge. The other three edges of the protective barrier are free so the provider may easily place them under the catheter.

Individually, the foam pad serves as skin protection as well as a resting platform on which the IV hub lies. The dressing is generally a plastic film sandwiched between a pair of juxtaposed backing pull tabs, adhered to the dressing adhesive, and a frame covering the perimeter of the film. The dressing body has a substantially rectangular portion of film with a transparent window and a pair of tabs extending therefrom, forming a flattened “A” shape. One of the tabs and some of the rectangular portion, once the backing is removed and the adhesive layer is displayed, is the first area to touch the skin to begin the securement process for the IV hub. The other tab and the remaining dressing, once the backing is removed and the adhesive surface is displayed, is the largest and strongest IV hub securement piece. This piece is the main securable feature that applies an adhesive covering over the IV catheter to secure its placement while also providing a protective covering. Once placed, the foam pad and the catheter hub are generally visible through the transparent window. When the elements are applied together, the overall device serves as a dressing for an IV catheter that not only secures the device but protects the skin. This dressing causes less discomfort, skin protection, and good IV hub securement all in one.

The materials of manufacture and the component sizes are not particularly limited. For example, any suitable polymeric foam may be used for the foam pad. The backing tabs may be made from any paper or plastic that is releasably attachable to the adhesive. The backing tabs may also be referred to herein as a removable release liner frame, a removable release liner tab, and two juxtaposed removable release liner strips.

To manufacture the all-in-one dressing, the foam rectangular piece may be applied to the back of the IV dressing approximately a centimeter below the catheter transparent window. The foam may be adhered hingedly by a fixed end to the adhesive surface of the film. The backing material applied to the dressing may be divided into two tabs to assist with placement and securement of the catheter.

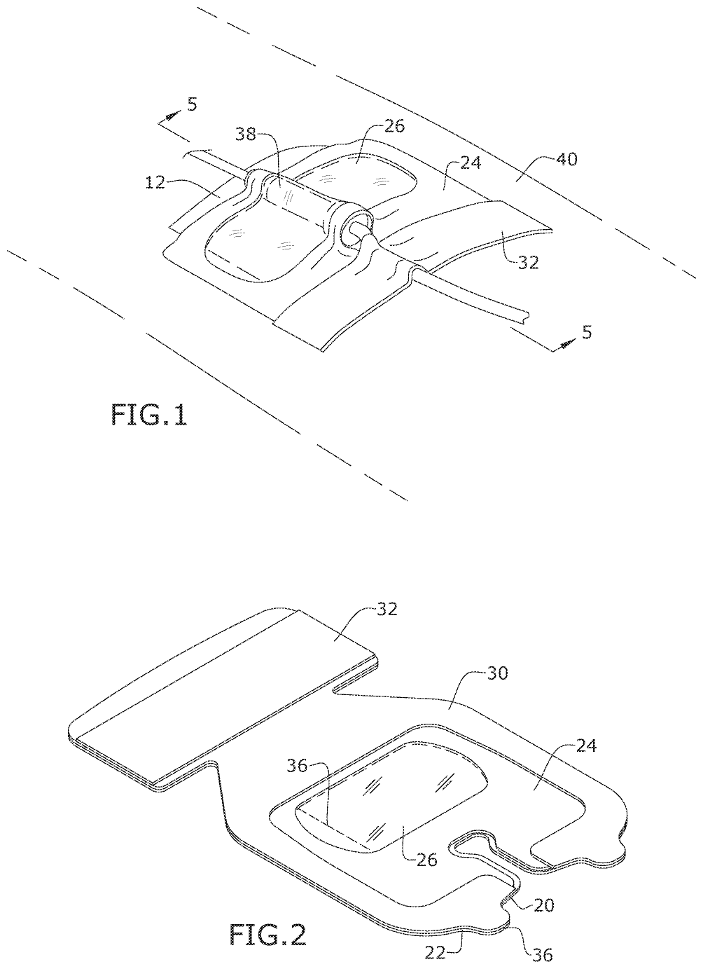

Referring to FIGS. 1 through 6, FIGS. 1 and 5 show an IV dressing 10 according to an embodiment of the present invention, without backing, applied to a patient 40. A foam pad 12 is adhered to the user's 40 skin underneath an IV hub 38. A film 24 with a window 26 is adhered over the IV hub 38, with an IV line emerging from the IV hub 38. The window 26 is positioned over the IV hub 38 for visibility. A adhesive strip, i.e., tape 32, holds the IV line in place on the patient's 40 skin.

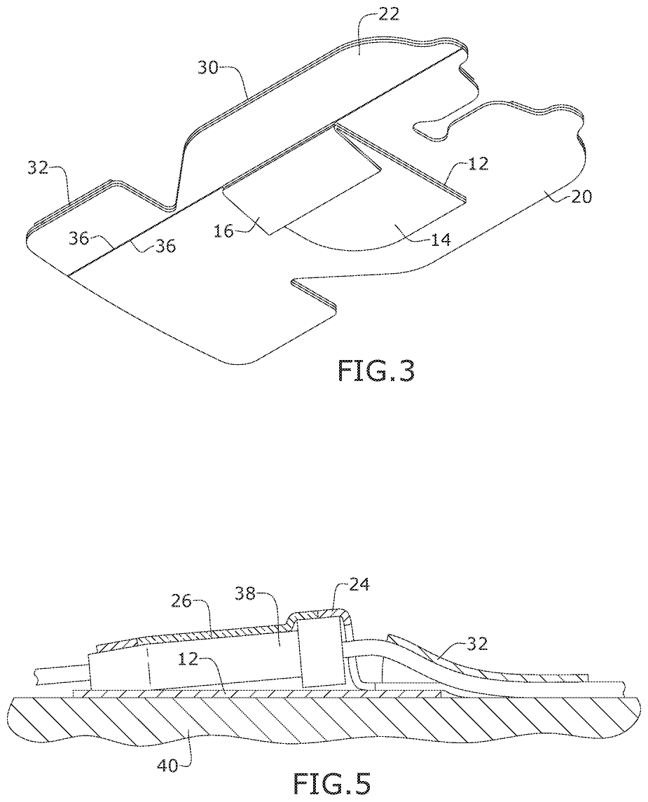

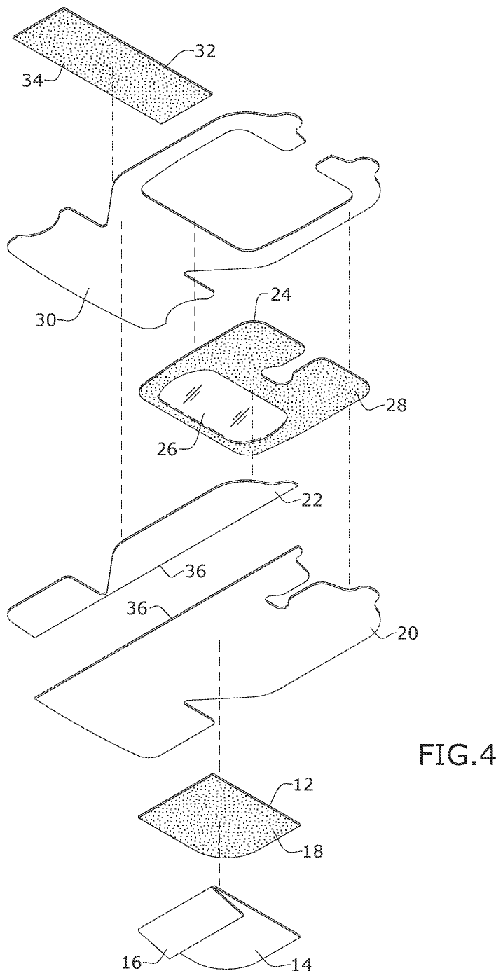

As shown in FIGS. 2 and 3, the IV dressing 10 comprises multiple layers of material including the film 24 and window 26 between a frame 30 of backing material on a first surface and, on an opposite second surface, a large pull tab backing 20 and a small pull tab backing 22, with a tab backing seam 36 visible through the window 26. The strip of tape 32 is attached to the frame 30. On the large pull tab backing 20, the foam pad 12 is adhered with a protective pad backing 14 and a pull tab 16. As shown in FIG. 4, the foam pad 12 has a first layer of adhesive 18, the film 24 has a second layer of adhesive 28, and the strip of tape 32 has a third layer of adhesive 34.

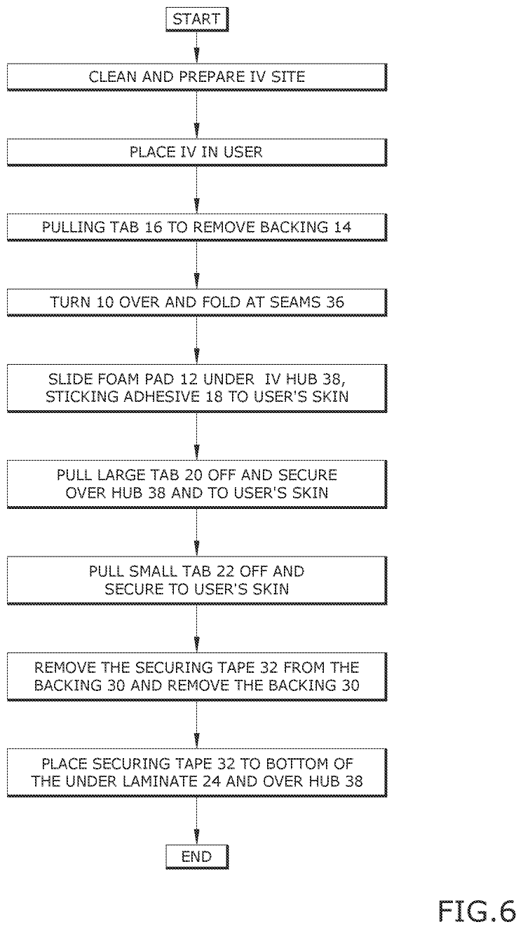

FIG. 6 illustrates a method of installation of the IV dressing 10 onto the patient 40. Once the IV site has been cleaned and prepared, the IV is placed and the line is connected. Backing 14 is removed by pulling tab 16 to expose adhesive 18, and the foam pad 12 is placed under the IV catheter hub 38 such that the adhesive 18 adheres to the patient 40. Tab 20 is removed to expose a first part of adhesive 28, and the dressing 10 is secured to the patient's skin 40 adjacent to the hub 38. Tab 22 is removed to expose the remaining part of adhesive 28, the line is centered in a slot or recessed portion between tab portions of the film 24, and the film 24 is adhered to the patient 40. In some embodiments, tab 22 may be removed before tab 20. The securing strip 32 is removed, and the frame 30 is removed, then the securing strip 32 is placed over the line with the adhesive 34 holding the line in place on the patient's 40 skin.

It should be understood, of course, that the foregoing relates to exemplary embodiments of the invention and that modifications may be made without departing from the spirit and scope of the invention as set forth in the following claims.

What is claimed is:

1. A skin protection and intravenous catheter securement dressing assembly, comprising:
a dressing body comprising a plastic film with a transparent window, a first adhesive surface, and a second surface, said plastic film having a recessed portion midway along one edge;
two juxtaposed removable release liner strips covering the first adhesive surface;
a foam pad with a second adhesive surface, a fixed end thereof hingedly connected to the first adhesive surface of the dressing;
a removable release liner tab covering the second adhesive surface;
a removable release liner frame covering a perimeter of the second surface; and
an adhesive strip removably attached to the removable release liner frame.

2. The skin protection and intravenous catheter securement dressing assembly of claim 1, wherein the fixed end is positioned such that at least part of the foam pad rests within an area visible through the transparent window.

3. The skin protection and intravenous catheter securement dressing assembly of claim 1, wherein the recessed portion is configured to accommodate a portion of a medical article extending from the transparent window on the first adhesive surface to the second surface.

4. The skin protection and intravenous catheter securement dressing assembly of claim 1, configured to accommodate a portion of a medical article between the foam pad and the transparent window.

5. A method for securing an intravenous catheter to a subject with a pressure relieving dressing, the method comprising:
preparing an intravenous (IV) site on the subject;
placing an IV catheter with hub at the IV site;
connecting an IV line to the hub;
providing the skin protection and intravenous catheter securement dressing assembly of claim 1;
removing the removable release liner tab;
placing the foam pad on the subject under the hub;
removing the two juxtaposed removable release liner strips sequentially;
centering the IV line in the recessed portion;
adhering the plastic film on the IV site over the hub;
removing the adhesive strip from the removable release liner frame;
removing the removable release liner frame from the plastic film; and
adhering the adhesive strip to the subject over the IV line.
